(19) United States
(12) Patent Application Publication
(10) Pub. No.: US 2012/0276108 A1
(43) Pub. Date: Nov. 1, 2012

(54) MONOSACCHARIDE-BASED COMPOUNDS FOR THE TREATMENT OF PROLIFERATIVE AND INFLAMMATORY DERMATOLOGICAL DISEASES

(75) Inventor: Waldemar Priebe, Houston, TX (US)

(73) Assignee: INTERTECH BIO, Houston, TX (US)

(21) Appl. No.: 13/347,968

(22) Filed: Jan. 11, 2012

Related U.S. Application Data
(60) Provisional application No. 61/431,731, filed on Jan. 11, 2011.

(51) Int. Cl. A61K 31/7024; A61K 31/7028; C07H 13/04; C07H 5/02; C07D 335/02; A61K 31/382; C07H 5/06; A61K 31/7008; A61K 31/70; C07H 13/06; C07H 7/02; C07H 99/00; C07H 5/10; C07H 9/04; A61P 17/00; A61P 17/06; A61P 17/08; A61P 17/10; A61P 29/00; A61P 17/02; A61P 35/00; A61P 37/06; A61P 31/04; A61K 39/395; A61K 31/7004; C07H 15/04

(52) U.S. Cl. 424/141.1; 536/18.2; 514/25; 536/119; 514/23; 536/122; 549/28; 514/432; 536/55.2; 514/62; 536/18.7; 536/54; 536/53; 536/120

(57) ABSTRACT

The present invention relates to compounds and methods which may be useful as inhibitors of glycolysis, inhibitors of protein glycosylation, anti-virals, and down-regulators of insulin receptor and IGF-1 receptor for the treatment or prevention of inflammatory dermatological diseases or proliferative dermatological diseases.

This application claims the benefit of priority of U.S. provisional application No. 61/431,731, filed Jan. 11, 2011, the disclosure of which is hereby incorporated by reference as if written herein in its entirety.

Disclosed herein are new monosaccharide-based compounds and compositions and their application as pharmaceuticals for the treatment of disease. Methods of inhibition of glycolysis, inhibition of protein glycosylation, anti-viral activity, and down-regulation of insulin receptor and IGF-1 receptor in a human or animal subject are also provided for the treatment of inflammatory dermatological diseases or proliferative dermatological diseases such as psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

Cells in eukaryotic organisms require energy to carry out cellular processes. Such energy is mainly stored in the phosphate bonds of adenosine 5′-triphosphate (“ATP”). Pathways that generate energy in eukaryotic organisms include: (1) glycolysis; (2) the Krebs Cycle (also referred to as the TCA cycle or citric acid cycle); and (3) oxidative phosphorylation. For ATP to be synthesized, carbohydrates are first hydrolyzed into monosaccharides (e.g., glucose), and lipids are hydrolyzed into fatty acids and glycerol. Likewise, proteins are hydrolyzed into amino acids. The energy in the chemical bonds of these hydrolyzed molecules are then released and harnessed by the cell to form ATP molecules through numerous catabolic pathways.

Reliance on glycolysis has been correlated with disease progression in cancer as well as a consistent and significant increase in activity of hexokinase, phosphofructokinase and pyruvate kinase. Hypoxia is found in certain solid cancers and has been linked to angiogenesis, differential tumor growth, malignant transformation, metastasis and treatment resistance.

Aerobic glycolysis is often enhanced by certain oncogenes through the increased expression of glucose transporters and glycolytic enzymes found on tumor cells.

Specifically, glucose is a simple sugar or monosaccharide, and the primary source of energy for animals. Glucose is an important sugar in human metabolism having a normal concentration of about 0.1% (usually 60 to 110 mgs per dl) in human blood except in persons suffering from diabetes. As a primary energy source, glucose requires no digestion.

The oxidation of glucose contributes to a series of complex biochemical reactions which provide the energy needed by cells. When oxidized (metabolized) in the body, glucose produces carbon dioxide, water and certain nitrogen compounds. Energy from glucose oxidation is used to convert ADP to adenosine 5′-triphosphate (“ATP”), a multifunctional nucleotide that is known as “molecular currency” of intracellular energy transfer.

ATP produced as an energy source during cellular respiration is consumed by different enzymes and cellular process including biosynthetic reactions, motility and cell division. For signal transduction pathways, ATP is the substrate by which kinases phosphorylate proteins and lipids and adenylate cyclase produces cyclic AMP.

ATP is an unstable molecule that tends to be hydrolyzed in water. Thus, if ATP and ADP are allowed to come into chemical equilibrium, almost all the ATP will be converted to ADP. Cells maintain ATP to ADP at a point ten orders of magnitude from equilibrium, with ATP concentrations a thousand fold higher than the concentration of ADP. This displacement from equilibrium means that the hydrolysis of ATP in the cell releases a lot of energy. Nicholls D. G. & Ferguson S J. (2002) Bioenergetics Academic Press 3rd Ed. ATP concentration inside the cell is typically 1-10 mM. Beis I., & Newsholme E. A. (1975) Biochem J 152, 23-32.

ATP is produced by redox reactions using simple sugars (e.g., glucose), complex sugars (carbohydrates), lipids, and proteins. For ATP to be synthesized, carbohydrates are hydrolyzed into simple sugars such as glucose, or fats (triglycerides) are hydrolyzed to give fatty acids and glycerol. Likewise, proteins are hydrolyzed to give amino acids. Cellular respiration is the process of oxidizing these hydrolyzed molecules to carbon dioxide to generate ATP. For instance, up to 36 molecules of ATP can be produced from a single molecule of glucose. Lodish, H. et al (2004) Molecular Cell Biology, 5th Ed. New York: WH Freeman. The three main pathways to generate energy in eukaryotic organisms are: glycolysis, the Krebs Cycle (also known as the citric acid cycle), and oxidative phosphorylation.

The main source of energy for living organisms is glucose. In breaking down glucose, the energy in the glucose molecule's chemical bonds is released and can be harnessed by the cell to form ATP molecules. The process by which this occurs consists of several stages. The first is called glycolysis (the prefix glyco refers to glucose, and lysis means to split), in which the glucose molecule is broken down into two smaller molecules called pyruvic acid. As further discussed below, the next stages are different for anaerobes and aerobes.

In glycolysis, glucose and glycerol are metabolized to pyruvate via the glycolytic pathway. In most organisms, glycolysis occurs in the cytosol. During this process, two ATP molecules are generated. Two molecules of NADH are also produced, which can be further oxidized via the electron transport chain and result in the generation of additional ATP molecules.

Glycolysis is the first stage in the release of energy from the glucose molecule. It occurs in the cytoplasm via many enzymes. Both aerobic and anaerobic organisms use glycolysis to break down glucose to pyruvate initially. After this stage, however, aerobic organisms utilize oxygen to obtain additional energy.

Glycolysis involves the breaking down of glucose into two smaller molecules of pyruvic acid, each pyruvic acid molecule having three carbon atoms, or half of the carbons in a glucose molecule. Noteworthy, for glycolysis to occur, two ATP molecules are necessary. The first ATP molecule releases a phosphate group which then joins to the glucose molecule to form glucose phosphate. Then, the second ATP molecule contributes a phosphate group, forming a molecule called fructose diphosphate. The fructose diphosphate molecule splits into two molecules of glyceraldehyde phosphate “PGAL.” Each PGAL molecule then releases electrons to a coenzyme NAD+ (nicotinamide adenine dinucleotide) and phosphate groups and energy to ADP.

As a result, two NAD+ molecules become NADH, and four molecules of ADP become ATP. In addition, the two molecules of PGAL have now become molecules of pyruvic acid, which has a molecular formula of C₃H₄O₃. Essentially, glycolysis requires an “investment” of two ATP molecules before it can begin. Since four ATP molecules are formed as products of the reaction, there is a net gain of two ATP molecules.

At this point in anaerobic organisms, pyruVIC acid (pyruvate) undergoes additional processing in order to obtain additional energy. These processes, however, are significantly less efficient than the processes which aerobes utilize: the Krebs cycle and the electron transport chain. Glycolysis occurs in the cytoplasm and involves many enzyme catalyzed steps that break down glucose (and other monosaccharides) into two pyruvate molecules.

In return, the pathway leads to the generation of a sum of two ATP molecules. The pyruvate molecules generated from the glycolytic pathway enter the mitochondria from the cytosol. The molecules are then converted to acetyl co-enzyme A (Acetyl-CoA) for entry into the Krebs cycle. The Krebs cycle consists of the bonding of acetyl coenzyme-A with oxaloacetate to form citrate. The formed citrate is then broken down through a series of enzyme-catalyzed steps to generate additional ATP molecules.

In addition to generating ATP, the catabolic processes in glycolysis and the Krebs cycle also generate electrons that become stored in the form of reduced co-enzymes, such as NADH and FADH2. These co-enzymes participate in oxidative phosphorylation, where their electrons pass through an electron transport chain across the mitochondrial membrane. During this process, the protons from NADH and FADH2 enter the mitochondrial intermembrane space. Consequently, the electron transport chain leads to the formation of a proton gradient within the intermembrane space. Finally, the protons flux from the intermembrane space to the mitochondrial matrix through specific proton channels that catalyze the synthesis of additional ATP molecules.

Like normal cells, cancer cells also utilize metabolic pathways to generate ATP. However, classic observations by Otto Warburg show that highly proliferative tumors utilize glycolysis for cellular energy production rather than oxidative phosphorylation or the Krebs cycle, even in the presence of normoxia or adequate amounts of oxygen (termed oxidative glycolysis or the “Warburg effect”). Energy Boost: The Warburg Effect Returns in a New Theory of Cancer, Journal of the National Cancer Institute, Vol. 96, No. 24, Dec. 15, 2004 at 1806. Hypoxia-inducible Factor 1 Activation by Aerobic Glycolysis Implicates the Warburg Effect in Carcinogenesis, J. Bio. Chem. Vol. 277, No. 26, 23111 (2002). Under such conditions, the tumor cells up-regulate the expression of both glucose transporters and glycolytic enzymes, in turn, favoring an increased uptake of glucose (as well as their analogs) as compared to normal cells in an aerobic environment. This tumor adaptive response appears to hold true for malignant gliomas as well.

Other prevalent changes that occur with the progression of malignant tumors is the activation of the PI-3KJ AKT pathway (typically by PTEN loss or through growth factor activity such as EGFR). This survival pathway activates a number of adaptive changes that include a stimulus for angiogenesis, inhibition of apoptosis, and metabolic shifts that promote activation of glycolysis and an increase in glucose uptake. Additionally, the malignant phenotype that up-regulates the glycolysis pathways are also induced by c-Myc, HIF-1a and STAT-3, all of which have been implicated in high-grade malignant transformation.

The aforementioned malignant transformations display a differential growth pattern. Namely, malignant tumors can grow in predominately hypoxic and mixed regions of variable degrees of normoxia. Relative hypoxic areas can be seen both in the center of the rapidly growing tumor mass, which often has regions of necrosis associated with this, as well as some relatively hypoxic regions within infiltrative components of the tumor as well. Accordingly, some of these relatively hypoxic regions may have cells that are cycling at a slower rate and may therefore be more resistant to many chemotherapy agents.

Many high-grade tumors are intrinsically resistant to conventional therapies. For instance, high-grade malignant tumors are highly angiogenic. In particular, most high-grade malignant tumors express large amounts of vascular endothelial growth factor (VEGF). AVASTIN®, a humanized monoclonal antibody against VEGF, has been used in combination with Irinotecan to treat patients with high grade gliomas. Results indicate very high response rates in over 60% of the treated patients (Society of Neuro-Oncology, 2005). This high response rate, however, is not translating into improved six month progression-free survival or overall survival at this point. Furthermore, many patients treated with AVASTIN® displayed markedly worsening non-contrasting infiltrative tumor disease progression, indicating “tumor escape”, or a shift of the growth phenotype to a predominately hypoxic pattern. (Conrad, C. A., et al., 2008 submitted).

Furthermore, many cancers are intrinsically resistant to conventional therapies and represent significant therapeutic challenges. In addition to the development of tumor resistance to treatments, another problem in treating malignant tumors is the toxicity of the treatment to normal tissues unaffected by disease. Often chemotherapy is targeted at killing rapidly-dividing cells regardless of whether those cells are normal or malignant. However, widespread cell death and the associated side effects of cancer treatments may not be necessary for tumor suppression if the growth control pathways of tumors can be disabled. For example, one approach is the use of therapy sensitization, i.e. using low dose of a standard treatment in combination with a drug that specifically targets crucial processes in the tumor cell, increasing the effects of the other drug.

Accordingly, the glycolytic pathway has become a potential target for the selective inhibition of many tumor cells. The inhibition of glycolysis would be selective for such tumor cells because normal cells in aerobic conditions would be able to survive such inhibition by generating energy through other pathways (e.g., the Krebs cycle, and oxidative phosphorylation). By contrast, when glycolysis is blocked in glycolytic tumor cells, the tumor cells would die because of an inability to utilize the aforementioned pathways.

However, current glycolytic inhibition approaches for cancer treatment present various challenges. For instance, many such treatments are not specific for the hypoxic environment of tumor cells. More importantly, current treatments are not selective inhibitors of glycolysis. Rather, such treatments can also target other pathways that are essential for normal cell function, such as glycosylation, where monosaccharides such as D-mannose form a part of oligosaccharides linked to proteins to form glycoproteins. Among other functions, glycoproteins are essential for maintaining the structural integrity of cell membranes. Thus, interference with glycosylation can have clinical consequences that may result in beneficial effects in the treatment of disease.

In addition to inhibiting glycolysis, compounds disclosed herein may also function as inhibitors of protein glycosylation, as anti-viral agents, and down-regulators of insulin receptor and IGF-1 receptor (Kang and Hwang, 2-Deoxyglucose: An anticancer and antiviral therapeutic, but not any more a low glucose mimetic Life Sciences 2006, 78, 1392-1399).

Novel compounds and pharmaceutical compositions, certain of which have been found to inhibit glycolysis, inhibit protein glycosylation, posess anti-viral activity, and down-regulate insulin receptor and IGF-1 receptor have been discovered, together with methods of synthesizing and using the compounds including methods for the treatment of inflammatory dermatological diseases and proliferative dermatological diseases in a patient by administering the compounds.

In certain embodiments of the present invention, compounds have structural Formula I:

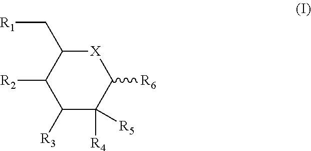

or a salt thereof, wherein:

X is selected from the group consisting of O and S;

R₁, R₂, R₃, and R₆ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, and carbamate, any of which may be optionally substituted;

R₄ and R₅ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, carbamate, alkyl, haloalkyl, perhaloalkyl, —N(R₇)OR₈, —ON(R₉)₂, —N(R₁₀)N(R₁₁)₂, any of which may be optionally substituted, or R₄ and R₅, taken together, are selected from the group consisting of ═N—OR₁₂ and ═N—N(R₁₃)₂; and

R₇, R₈, R₉, R₁₀, R₁₁, R₁₂, and R₁₃ are each independently selected from the group consisting of hydrogen and alkyl, wherein said alkyl may be optionally substituted.

Certain compounds disclosed herein may possess useful glycolysis inhibiting activity, protein glycosylation inhibiting activity, anti-viral activity, and insulin receptor and IGF-1 receptor down-regulating activity, and may be used in the treatment or prophylaxis of a disease or condition in which glycolysis, glycosylation, viral infection, insulin receptors, or IGF-1 receptors play an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting glycolysis, inhibiting protein glycosylation, suppressing viral activity, and down-regulating insulin receptor and IGF-1 receptor expression. Other embodiments provide methods for treating an inflammatory dermatological disease or a proliferative dermatological disease in a patient in need of such treatment, comprising administering to said patient a therapeutically effective amount of a compound or composition according to the present invention. Also provided is the use of certain compounds disclosed herein for use in the manufacture of a medicament for the treatment of an inflammatory dermatological disease or a proliferative dermatological disease.

In certain embodiments, the compounds have structural Formula II:

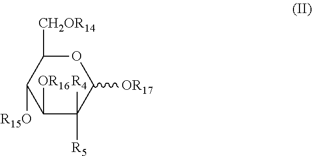

or a salt thereof, wherein:

R₁₄, R₁₅, R₁₆, and R₁₇ are independently selected from the group consisting of hydrogen, COCH₃, COCH₂CH₃, and COCH₂CH₂CH₃; and

R₄ and R₅ are independently selected from the group consisting of hydrogen, Cl, Br, I, ¹⁸F, and ¹⁹F.

In further embodiments, R₄ and R₅ are independently selected from the group consisting of hydrogen, ¹⁸F, and ¹⁹F.

In further embodiments, R₁₄, R₁₅, R₁₆, and R₁₇ are independently selected from the group consisting of hydrogen and COCH₃.

In certain embodiments, the compounds have structural Formula III or structural Formula IV:

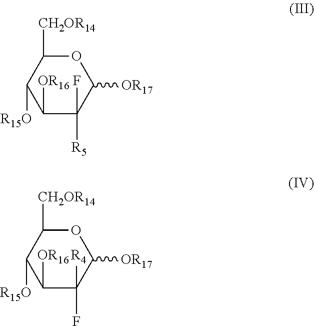

or a salt thereof, wherein:

R₁₄, R₁₅, R₁₆, and R₁₇ are independently selected from the group consisting of hydrogen, COCH₃, COCH₂CH₃, and COCH₂CH₂CH₃;

R₄ and R₅ are independently selected from the group consisting of alkyl, lower alkyl, substituted alkyl, cycloalkyl, hydroxyl, alkoxy, acyl, alkenyl, alkylene, alkylamino, alkylthio, alkylidene, alkynyl, amido, carbamoyl, acylamino, carbamate, O-carbamyl, N-carbamyl, carbonyl, carboxy, carboxylate, ester, ether, halogen, haloalkoxy, haloalkyl, heteroalkyl, hydrazinyl, hydroxyalkyl, isocyanato, isothiocyanato, mercaptyl, nitro, oxy, NH₂, NR₁₈R₁₉, and NHCOR₂₀;

R₁₈ and R₁₉ are selected from the group consisting of hydrogen, alkyl, lower alkyl, substituted alkyl, cycloalkyl, acyl, alkenyl, alkylene, alkylamino, alkylthio, alkylidene, alkynyl, amido, haloalkyl, heteroalkyl, hydrazinyl, and hydroxyalkyl; and

R₂₀ is selected from the group consisting of hydrogen, lower alkyl, substituted lower alkyl, alkenyl, alkylene, alkylamino, alkylthio, alkylidene, alkynyl, amido, carbamoyl, haloalkyl, and heteroalkyl.

In further embodiments, R₁₄, R₁₅, R₁₆, and R₁₇ are hydrogen.

In certain embodiments, the compounds have structural Formula V:

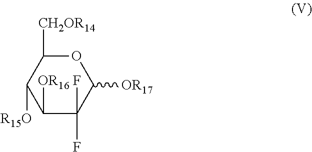

or a salt thereof, wherein:

R₁₄, R₁₅, R₁₆, and R₁₇ are independently selected from the group consisting of hydrogen, COCH₃, COCH₂CH₃, and COCH₂CH₂CH₃.

In certain embodiments, a compound is selected from the group consisting of Examples 1 to 83.

In certain embodiments, a compound has the following structural formula:

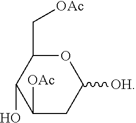

In certain embodiments, wherein said dermatological disease is an inflammatory dermatological disease or a proliferative dermatological disease.

In certain embodiments, an inflammatory dermatological disease or proliferative dermatological disease is selected from the group consisting of psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

In certain embodiments, a compound of the present invention is administered with another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STAT5 inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

In certain embodiments, disclosed herein is a topical pharmaceutical composition for the treatment of a dermatological disease.

In certain embodiments, said topical pharmaceutical composition is a gel, liniment, lotion, cream, ointment, or paste.

In certain embodiments, a topical pharmaceutical composition comprises a compound of the present invention together with another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STAT5 inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)-group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′-group, with R and R′ as defined herein.

The term “carbonyl,” as used herein, when alone includes formyl [—C(O)H] and in combination is a —C(O)-group.

The term “carboxyl” or “carboxy,” as used herein, refers to —C(O)OH or the corresponding “carboxylate” anion, such as is in a carboxylic acid salt. An

“O-carboxy” group refers to a RC(O)O-group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like.

“Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, said heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each said heteroatom may be independently selected from the group consisting of nitrogen, oxygen, and sulfur In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS-group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′-group with R and R′ as defined herein.

The term “S-sulfonamido” refers to a —S(═O)₂NRR′, group, with R and R′ as defined herein.

The terms “thia” and “thio,” as used herein, alone or in combination, refer to a —S-group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)-group.

The term “N-thiocarbamyl” refers to an ROC(S)NR′-group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR-group with X is a halogen and R as defined herein.

The term “trihalomethanesulfonyl” refers to a X₃CS(O)₂-group where X is a halogen.

The term “trihalomethoxy” refers to a X₃CO-group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“Glycolysis inhibitor” is used herein to refer to a compound that exhibits an IC₅₀ with respect to glycolytic activity or anticancer activity of no more than about 100 μM and more typically not more than about 50 μM, as measured in the assay name described generally hereinbelow. “IC₅₀” is that concentration of inhibitor which reduces the activity of glycolysis to half-maximal level. Certain compounds disclosed herein have been discovered to exhibit inhibition against glycolysis. In certain embodiments, compounds will exhibit an IC₅₀ with respect to glycolysis of no more than about 10 μM; in further embodiments, compounds will exhibit an IC₅₀ with respect to glycolysis of no more than about 5 μM; in yet further embodiments, compounds will exhibit an IC₅₀ with respect to glycolysis of not more than about 1 μM; in yet further embodiments, compounds will exhibit an IC₅₀ with respect to glycolysis of not more than about 200 nM, as measured in the glycolysis assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation or proliferation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

Gels for topical or transdermal administration may comprise, generally, a mixture of volatile solvents, nonvolatile solvents, and water. In certain embodiments, the volatile solvent component of the buffered solvent system may include lower (C1-C6) alkyl alcohols, lower alkyl glycols and lower glycol polymers. In further embodiments, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. In certain embodiments, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess may result in a lack of bioavailability due to poor release of drug from solvent mixture. The buffer component of the buffered solvent system may be selected from any buffer commonly used in the art; in certain embodiments, water is used. A common ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops may comprise sterile aqueous or oily solutions or suspensions and may be prepared by dissolving the active ingredient in a suitable aqueous solution of a bactericidal and/or fungicidal agent and/or any other suitable preservative, and, in certain embodiments, including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with cell differentiating agents, anti-proliferative agents, mitochondrial inhibitors, topical steroids, immunosuppressive compounds, JAK2 inhibitors, JAK3 inhibitors, STAT3 inhibitors, STATS inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, D-mannose, and combinations thereof.

In certain embodiments, cell differentiating agents include, but are not limited to retinoic acid, retinol, retinal, isotretinoin alitretinoin, etretinate, acitretin, tazarotene, bexarotene, adapalene, vitamin D, alfacalcidol (1-hydroxycholecalciferol), calcitriol (1,25-dihydroxycholecalciferol), cholecalciferol (vitamin D3), dihydrotachysterol (DHT) and ergocalciferol (vitamin D2), phorbol esters, and 12-O-tetradecanoylphorbol-13-acetate.

In certain embodiments, mitochondrial inhibitors include, but are not limited to, anthraline, dithranol, chrysarobin, and coal tar.

In certain embodiments, topical steroids include, but are not limited to, clobetasol propionate, betamethasone, betamethasone dipropionate, halobetasol propionate, fluocinonide, diflorasone diacetate, mometasone furoate, halcinonide, desoximetasone, fluticasone propionate, flurandrenolide, triamcinolone acetonide, fluocinolone acetonide, hydrocortisone, hydrocortisone valerate, prednicarbate, desonide, and alclometasone dipropionate.

In certain embodiments, immunosuppressive compounds include, but are not limited to, fingolimod, cyclosporine A, azathioprine, dexamethasone, tacrolimus, sirolimus, pimecrolimus, mycophenolate salts, everolimus, basiliximab, daclizumab, anti-thymocyte globulin, anti-lymphocyte globulin, CTLA4IgG, D-penicillamine, gold salts, hydroxychloroquine, leflunomide, methotrexate, minocycline, sulfasalazine, cyclophosphamide, etanercept, infliximab, adalimumab, anakinra, rituximab, and abatacept.

In certain embodiments, JAK2 inhibitors include, but are not limited to, INCBI8424.

In certain embodiments, JAK3 inhibitors include, but are not limited to, CP-690,550.

In certain embodiments, STAT3 inhibitors include, but are not limited to, WP1066, WP1193, WP1130, and WP1220/MOL4239.

In certain embodiments, STATS inhibitors include, but are not limited to, WP1220/MOL4239.

In certain embodiments, parathyroid hormone-related protein (PTHrP) agonists include, but are not limited to, PTH(1-34).

In certain embodiments, cell adhesion blockers include, but are not limited to, bimosiamose.

In certain embodiments, non-steroidal anti-inflammatory agents include, but are not limited to, aceclofenac, acemetacin, amoxiprin, aspirin, azapropazone, benorilate, bromfenac, carprofen, celecoxib, choline magnesium salicylate, diclofenac, diflunisal, etodolac, etoracoxib, faislamine, fenbuten, fenoprofen, flurbiprofen, ibuprofen, indometacin, ketoprofen, ketorolac, lornoxicam, loxoprofen, lumiracoxib, meloxicam, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinprazone, suprofen, tenoxicam, tiaprofenic acid, and tolmetin.

In certain embodiments, antibacterial agents include, but are not limited to, amikacin, amoxicillin, ampicillin, arsphenamine, azithromycin, aztreonam, azlocillin, bacitracin, carbenicillin, cefaclor, cefadroxil, cefamandole, cefazolin, cephalexin, cefdinir, cefditorin, cefepime, cefixime, cefoperazone, cefotaxime, cefoxitin, cefpodoxime, cefprozil, ceftazidime, ceftibuten, ceftizoxime, ceftriaxone, cefuroxime, chloramphenicol, cilastin, ciprofloxacin, clarithromycin, clindamycin, cloxacillin, colistin, dalfopristan, demeclocycline, dicloxacillin, dirithromycin, doxycycline, erythromycin, enafloxacin, ertepenem, ethambutol, flucloxacillin, fosfomycin, furazolidone, gatifloxacin, geldanamycin, gentamicin, herbimicin, imipenem, isoniazide, kanamicin, levofloxacin, linezolid, lomefloxacin, loracarbef, mafenide, moxifloxacin, meropenem, metronidazole, mezlocillin, minocycline, mupirozin, nafcillin, neomycin, netilmicin, nitrofurantoin, norfloxacin, ofloxacin, oxytetracycline, penicillin, piperacillin, platensimycin, polymixin B, prontocil, pyrazinamide, quinupristine, rifampin, retapamulin, roxithromycin, spectinomycin, streptomycin, sulfacetamide, sulfamethizole, sulfamethoxazole, teicoplanin, telithromycin, tetracycline, ticarcillin, tobramycin, trimethoprim, troleandomycin, trovafloxacin, and vancomycin.

In certain embodiments, alkylating agents include, but are not limited to, chlorambucil, chlormethine, cyclophosphamide, ifosfamide, melphalan, carmustine, fotemustine, lomustine, streptozocin, carboplatin, cisplatin, oxaliplatin, BBR3464, busulfan, dacarbazine, procarbazine, temozolomide, thioTEPA, and uramustine.

In certain embodiments, anti-metabolite agents include, but are not limited to, aminopterin, methotrexate, pemetrexed, raltitrexed, cladribine, clofarabine, fludarabine, mercaptopurine, pentostatin, tioguanine, cytarabine, fluorouracil, floxuridine, tegafur, carmofur, capecitabine and gemcitabine.

In certain embodiments, mitotic inhibitors include, but are not limited to, docetaxel, paclitaxel, vinblastine, vincristine, vindesine, and vinorelbine.

In certain embodiments, tyrosine kinase inhibitors include, but are not limited to, dasatinib, erlotinib, gefitinib, imatinib, lapatinib, nilotinib, sorafenib, and sunitinib.

In certain embodiments, topoisomerase inhibitors include, but are not limited to, etoposide, etoposide phosphate, teniposide, camptothecin, topotecan, and irinotecan.

In certain embodiments, cancer immunotherapy monoclonal antibodies include, but are not limited to, rituximab, alemtuzumab, bevacizumab, cetuximab, gemtuzumab, panitumumab, tositumomab, and trastuzumab.

In certain embodiments, anti-tumor antibiotic agents include, but are not limited to, anthracycline, mithramycin, fludarabine, gemcetobine, daunorubicin, doxorubicin, epirubicin, idarubicin, mitoxantrone, valrubicin, actinomycin, bleomycin, mitomycin, plicamycin, and hydroxyurea.

In certain embodiments, anti-cancer agents include, but are not limited to, amsacrine, asparaginase, altretamine, hydroxycarbamide, lonidamine, pentostatin, miltefosine, masoprocol, estramustine, tretinoin, mitoguazone, topotecan, tiazofurine, irinotecan, alitretinoin, mitotane, pegaspargase, bexarotene, arsenic trioxide, imatinib, denileukin diftitox, bortezomib, celecoxib, and anagrelide.

In certain embodiments, autophagy-inducing agents include, but are not limited to, rapamycin, concanavalin A, eEF-2 kinase inhibitors, and SAHA.

In certain embodiments, anti-psoriasis drugs include, but are not limited to, AEB071, AlN457, UO267, BIRT 2584, SRT2104, ILV-095, PH-10, tetrathiomolybdate, ASP015K, VB-201, RWJ-445380, botulinum toxin, CF101, CNTO 1275, CTA018, ILV-094, LY2439821, BT061, AMG 827, PTH (1-34), QRX-101, CNTO 1959, CTLA4Ig, AMG 139, and MK-0873.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating inflammatory dermatological diseases or proliferative dermatological diseases in a human or animal subject in need of such treatment comprising administering to said subject an amount of a compound disclosed herein effective to reduce or prevent said disease in the subject, in combination with at least one additional agent for the treatment of said disease that is known in the art. In a related aspect, certain embodiments provide therapeutic compositions comprising at least one compound disclosed herein in combination with one or more additional agents for the treatment of inflammatory dermatological diseases or proliferative dermatological diseases.

Specific diseases to be treated by the compounds, compositions, and methods disclosed herein include, but are not limited to, inflammatory dermatological diseases, proliferative dermatological diseases, psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### General Synthetic Methods for Preparing Compounds

The compounds disclosed herein can be synthesized according to the procedures described in WO 2010005799 (paragraphs [0086]-[0145]); WO 2009108926 (paragraphs [0173]-[0185]); WO 2008131024 (paragraphs [0067]-[0072]); US 20100152121 (paragraphs [0067]-[0083]); U.S. Pat. No. 7,160,865 (columns 11-13); and U.S. Pat. No. 6,979,675 (columns 28-29), the disclosures of which are hereby incorporated by reference as if written herein in their entireties.

The invention is further illustrated by the following examples.

### EXAMPLE 1

### (4R,5S,6R)-4,5-dihydroxy-6-(hydroxymethyl)tetrahydro-2H-pyran-2-yl acetate

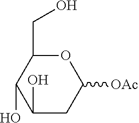

### EXAMPLE 2

### (4R,5S,6R)-5-hydroxy-6-(hydroxymethyl)tetrahydro-2H-pyran-2,4-diyl diacetate

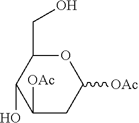

### EXAMPLE 3

### ((2R,3S,4R)-6-acetoxy-3,4-dihydroxytetrahydro-2H-pyran-2-yl)methyl acetate

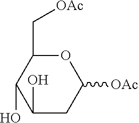

### EXAMPLE 4

### ((2R,3S,4R)-3-acetoxy-4,6-dihydroxytetrahydro-2H-pyran-2-yl)methyl acetate

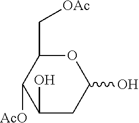

### EXAMPLE 5

### ((2R,3S,4R)-4-acetoxy-3,6-dihydroxytetrahydro-2H-pyran-2-yl)methyl acetate

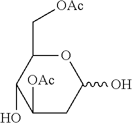

### EXAMPLE 6

### ((2R,3S,4R)-3,4,6-trihydroxytetrahydro-2H-pyran-2-yl)methyl acetate

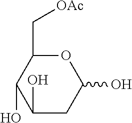

### EXAMPLE 7

### (2R,3S,4R)-4,6-dihydroxy-2-(hydroxymethyl)tetrahydro-2H-pyran-3-yl acetate

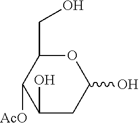

### EXAMPLE 8

### (4R,5S,6R)-4-hydroxy-6-(hydroxymethyl)tetrahydro-2H-pyran-2,5-diyl diacetate

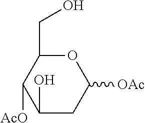

### EXAMPLE 9

### (2R,3S,4R)-6-hydroxy-2-(hydroxymethyl)tetrahydro-2H-pyran-3,4-diyl diacetate

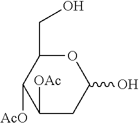

### EXAMPLE 10

### (4R,5S,6R)-6-(acetoxymethyl)-5-hydroxytetrahydro-2H-pyran-2,4-diyl diacetate

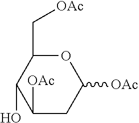

### EXAMPLE 11

### (4R,5S,6R)-6-(hydroxymethyl)tetrahydro-2H-pyran-2,4,5-triyl triacetate

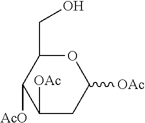

### EXAMPLE 12

### (4R,5S,6R)-6-(acetoxymethyl)tetrahydro-2H-pyran-2,4,5-triyl triacetate

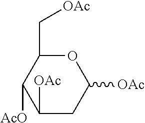

### EXAMPLE 13

### (4S,5S,6R)-3,3-difluoro-6-(hydroxymethyl)tetrahydro-2H-pyran-2,4,5-triol (2-Deoxy-2,2-difluoro-D-arabino-hexopyranose (DFG))

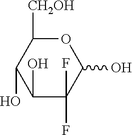

### EXAMPLE 14

### (3R,4S,5S,6R)-6-(hydroxymethyl)-3-iodotetrahydro-2H-pyran-2,4,5-triol (2-Deoxy-2-iodo-D-glucose)

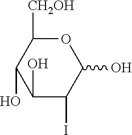

### EXAMPLE 15

### (3S,4S,5S,6R)-6-(hydroxymethyl)-3-iodotetrahydro-2H-pyran-2,4,5-triol (2-Deoxy-2-iodo-D-mannose)

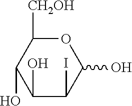

### EXAMPLE 16

### (3R,4S,5R,6R)-6-(hydroxymethyl)-3-iodotetrahydro-2H-pyran-2,4,5-triol (2-Deoxy-2-iodo-D-galactose)

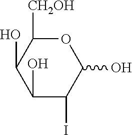

### EXAMPLE 17

### (3S,4S,5R,6R)-6-(hydroxymethyl)-3-iodotetrahydro-2H-pyran-2,4,5-triol (2-Deoxy-2-iodo-D-talose)

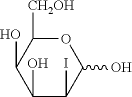

### EXAMPLE 18

### (4R,5S,6R)-6-(hydroxymethyl)tetrahydro-2H-pyran-2,4,5-triol (2-deoxy-D-glucose)

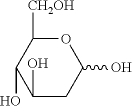

### EXAMPLE 19

### (3S,4S,5S,6R)-3-fluoro-6-(hydroxymethyl)tetrahydro-2H-pyran-2,4,5-triol (2-Deoxy-2-fluoro-D-mannose)

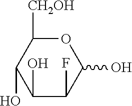

### EXAMPLE 20

### (3R,4S,5S,6R)-3-fluoro-6-(hydroxymethyl)tetrahydro-2H-pyran-2,4,5-triol (2-Deoxy-2-fluoro-D-glucose)

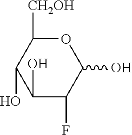

### EXAMPLE 21

### (3R,4S,5S,6R)-3-chloro-6-(hydroxymethyl)tetrahydro-2H-pyran-2,4,5-triol (2-Deoxy-2-chloro-D-glucose)

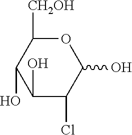

### EXAMPLE 22

### (3S,4S,5S,6R)-3-chloro-6-(hydroxymethyl)tetrahydro-2H-pyran-2,4,5-triol (2-Deoxy-2-chloro-D-mannose)

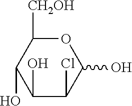

### EXAMPLE 23

### (3R,4S,5S,6R)-3-bromo-6-(hydroxymethyl)tetrahydro-2H-pyran-2,4,5-triol (2-Deoxy-2-bromo-D-glucose)

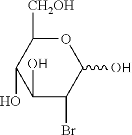

### EXAMPLE 24

### (3S,4S,5S,6R)-3-bromo-6-(hydroxymethyl)tetrahydro-2H-pyran-2,4,5-triol (2-Deoxy-2-bromo-D-mannose)

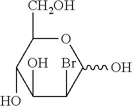

### EXAMPLE 25

### (3R,4S,5S,6R)-6-methyltetrahydro-2H-pyran-2,3,4,5-tetraol (6-Deoxy-D-glucose)

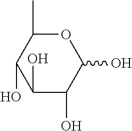

### EXAMPLE 26

### (3R,4S,5S,6S)-6-(fluoromethyl)tetrahydro-2H-pyran-2,3,4,5-tetraol (6-Deoxy-6-fluoro-D-glucose)

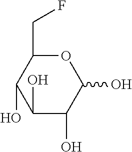

### EXAMPLE 27

### (3R,4S,5S,6R)-6-(methoxymethyl)tetrahydro-2H-pyran-2,3,4,5-tetraol (6-O-Methyl-D-glucose)

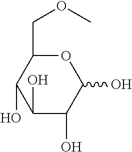

### EXAMPLE 28

### (3R,4S,5S,6R)-2-fluoro-6-(hydroxymethyl)tetrahydro-2H-pyran-3,4,5-triol (D-Glucosyl fluoride)

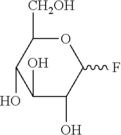

### EXAMPLE 29

### (2R,3S,4R,5S)-2-(hydroxymethyl)tetrahydro-2H-pyran-3,4,5-triol (1-Deoxy-D-glucose)

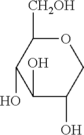

### EXAMPLE 30

### (3R,4R,5S,6R)-5-fluoro-6-(hydroxymethyl)tetrahydro-2H-pyran-2,3,4-triol (4-Fluoro-D-glucose)

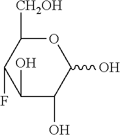

### EXAMPLE 31

### (3R,4S,6S)-6-(hydroxymethyl)tetrahydro-2H-pyran-2,3,4-triol (4-Deoxy-D-glucose)

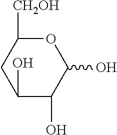

### EXAMPLE 32

### (3S,4S,5R,6R)-4-fluoro-6-(hydroxymethyl)tetrahydro-2H-pyran-2,3,5-triol (3-Fluoro-D-glucose)

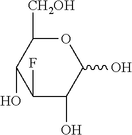

### EXAMPLE 33

### (3R,5S,6R)-6-(hydroxymethyl)tetrahydro-2H-pyran-2,3,5-triol (3-Deoxy-D-glucose)

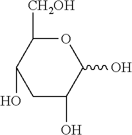

### EXAMPLE 34

### (3R,4S,5S,6S)-6-(mercaptomethyl)tetrahydro-2H-pyran-2,3,4,5-tetraol (6-Thio-D-glucose)

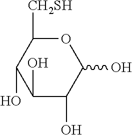

### EXAMPLE 35

### (3R,4S,5S,6R)-6-(hydroxymethyl)tetrahydro-2H-thiopyran-2,3,4,5-tetraol (5-Thio-D-glucose)

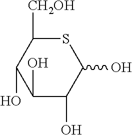

### EXAMPLE 36

### ((2R,3S,4S,5R)-3,4,5,6-tetrahydroxytetrahydro-2H-pyran-2-yl)methyl pentanoate

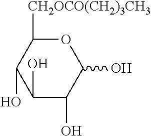

### EXAMPLE 37

### ((2R,3S,4S,5R)-3,4,5,6-tetrahydroxytetrahydro-2H-pyran-2-yl)methyl tetradecanoate

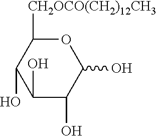

### EXAMPLE 38

### ((2R,3S,4S,5R)-3,4,5,6-tetrahydroxytetrahydro-2H-pyran-2-yl)methyl palmitate

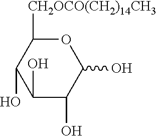

### EXAMPLE 39

### (2R,3S,4R,5R)-4,5,6-trihydroxy-2-(hydroxymethyl)tetrahydro-2H-pyran-3-yl pentanoate

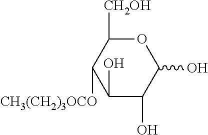

### EXAMPLE 40

### (2S,3S,4S,5R)-tridecyl 4,5,6-trihydroxy-2-(hydroxymethyl)tetrahydro-2H-pyran-3-carboxylate

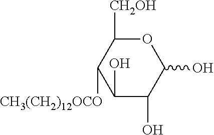

### EXAMPLE 41

### (2R,3S,4R,5R)-4,5,6-trihydroxy-2-(hydroxymethyl)tetrahydro-2H-pyran-3-yl palmitate

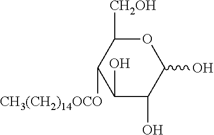

### EXAMPLE 42

### (3R,4S,5R,6R)-2,3,5-trihydroxy-6-(hydroxymethyl)tetrahydro-2H-pyran-4-yl pentanoate

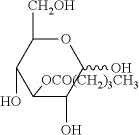

### EXAMPLE 43

### (3R,4S,5R,6R)-2,3,5-trihydroxy-6-(hydroxymethyl)tetrahydro-2H-pyran-4-yl tetradecanoate

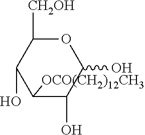

### EXAMPLE 44

### (3R,4S,5R,6R)-2,3,5-trihydroxy-6-(hydroxymethyl)tetrahydro-2H-pyran-4-yl palmitate

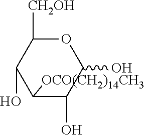

### EXAMPLE 45

### (3R,4S,5S,6R)-3,4,5-trihydroxy-6-(hydroxymethyl)tetrahydro-2H-pyran-2-yl pentanoate

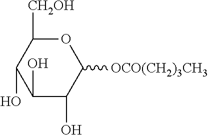

### EXAMPLE 46

### (3R,4S,5S,6R)-3,4,5-trihydroxy-6-(hydroxymethyl)tetrahydro-2H-pyran-2-yl tetradecanoate

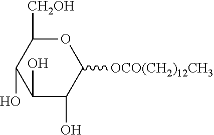

### EXAMPLE 47

### (3R,4S,5S,6R)-3,4,5-trihydroxy-6-(hydroxymethyl)tetrahydro-2H-pyran-2-yl palmitate

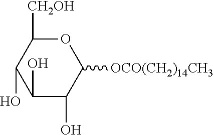

### EXAMPLE 48

### (3R,4R,5S,6R)-3-amino-6-(hydroxymethyl)tetrahydro-2H-pyran-2,4,5-triol (2-Glucosamine)

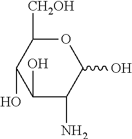

### EXAMPLE 49

### (3R,4R,5R,6R)-3-amino-6-(hydroxymethyl)tetrahydro-2H-pyran-2,4,5-triol (2-Galactosamine)

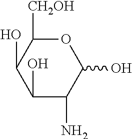

### EXAMPLE 50

### (3S,4R,5S,6R)-3-amino-6-(hydroxymethyl)tetrahydro-2H-pyran-2,4,5-triol (2-Mannosamine)

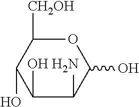

### EXAMPLE 51

### (3S,4S,5S,6R)-3-fluoro-6-(hydroxymethyl)tetrahydro-2H-pyran-2,4,5-triol (2-Deoxy-2-fluoro-D-mannose)

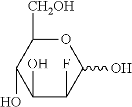

### EXAMPLE 52

### (4R,5R,6R)-6-(hydroxymethyl)tetrahydro-2H-pyran-2,4,5-triol (2-Deoxy-D-galactose)

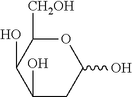

### EXAMPLE 53

### (3R,4S,5R,6R)-3-fluoro-6-(hydroxymethyl)tetrahydro-2H-pyran-2,4,5-triol (2-Deoxy-2-fluoro-D-galactose)

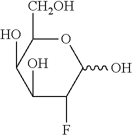

### EXAMPLE 54

### N-((3R,4R,5S,6R)-2,4,5-trihydroxy-6-(hydroxymethyl)tetrahydro-2H-pyran-3-yl)methanesulfonamide

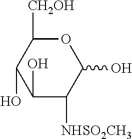

### EXAMPLE 55

### N-((3S,4R,5S,6R)-2,4,5-trihydroxy-6-(hydroxymethyl)tetrahydro-2H-pyran-3-yl)methanesulfonamide

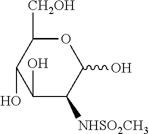

### EXAMPLE 56

### (3R,4R,5S,6R)-6-(hydroxymethyl)-3-methyltetrahydro-2H-pyran-2,4,5-triol

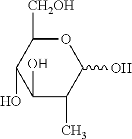

### EXAMPLE 57

### (3R,4R,5S,6R)-3-(fluoromethyl)-6-(hydroxymethyl)tetrahydro-2H-pyran-2,4,5-triol

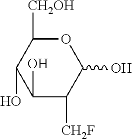

### EXAMPLE 58

### (3R,4R,5S,6R)-3-(difluoromethyl)-6-(hydroxymethyl)tetrahydro-2H-pyran-2,4,5-triol

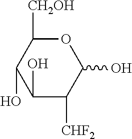

### EXAMPLE 59

### (3R,4R,5S,6R)-6-(hydroxymethyl)-3-(trifluoromethyl)tetrahydro-2H-pyran-2,4,5-triol

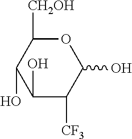

### EXAMPLE 60

### (3S,4R,5S,6R)-6-(hydroxymethyl)-3-methyltetrahydro-2H-pyran-2,4,5-triol

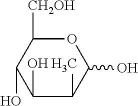

### EXAMPLE 61

### (3S,4R,5S,6R)-3-(fluoromethyl)-6-(hydroxymethyl)tetrahydro-2H-pyran-2,4,5-triol

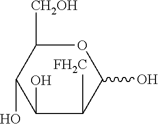

### EXAMPLE 62

### (3S,4R,5S,6R)-3-(difluoromethyl)-6-(hydroxymethyl)tetrahydro-2H-pyran-2,4,5-triol

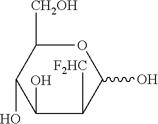

### EXAMPLE 63

### (3S,4R,5S,6R)-6-(hydroxymethyl)-3-(trifluoromethyl)tetrahydro-2H-pyran-2,4,5-triol

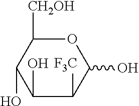

### EXAMPLE 64

### (3S,4S,5S,6R)-6-(hydroxymethyl)-3-methyltetrahydro-2H-pyran-2,3,4,5-tetraol

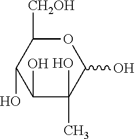

### EXAMPLE 65

### (3S,4S,5S,6R)-3-(fluoromethyl)-6-(hydroxymethyl)tetrahydro-2H-pyran-2,3,4,5-tetraol

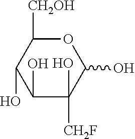

### EXAMPLE 66

### (3S,4S,5S,6R)-3-(difluoromethyl)-6-(hydroxymethyl)tetrahydro-2H-pyran-2,3,4,5-tetraol

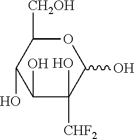

### EXAMPLE 67

### (3S,4S,5S,6R)-6-(hydroxymethyl)-3-(trifluoromethyl)tetrahydro-2H-pyran-2,3,4,5-tetraol

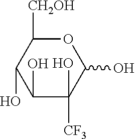

### EXAMPLE 68

### (3R,4S,5S,6R)-6-(hydroxymethyl)-3-methyltetrahydro-2H-pyran-2,3,4,5-tetraol

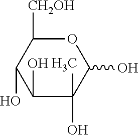

### EXAMPLE 69

### (3R,4S,5S,6R)-3-(fluoromethyl)-6-(hydroxymethyl)tetrahydro-2H-pyran-2,3,4,5-tetraol

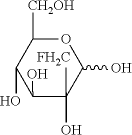

### EXAMPLE 70

### (3R,4S,5S,6R)-3-(difluoromethyl)-6-(hydroxymethyl)tetrahydro-2H-pyran-2,3,4,5-tetraol

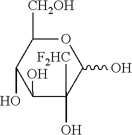

### EXAMPLE 71

### (3R,4S,5S,6R)-6-(hydroxymethyl)-3-(trifluoromethyl)tetrahydro-2H-pyran-2,3,4,5-tetraol

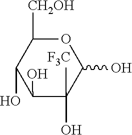

### EXAMPLE 72

### N-((3R,4R,5S,6R)-2,4,5-trihydroxy-6-(hydroxymethyl)tetrahydro-2H-pyran-3-yl)acetamide

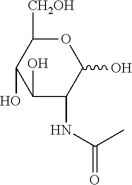

### EXAMPLE 73

### (3R,4S,5S,6R)-3-(aminooxy)-6-(hydroxymethyl)tetrahydro-2H-pyran-2,4,5-triol

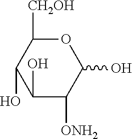

### EXAMPLE 74

### (3S,4S,5S,6R)-3-(aminooxy)-6-(hydroxymethyl)tetrahydro-2H-pyran-2,4,5-triol

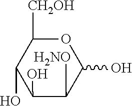

### EXAMPLE 75

### (3R,4R,5S,6R)-3-(hydroxyamino)-6-(hydroxymethyl)tetrahydro-2H-pyran-2,4,5-triol

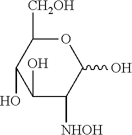

### EXAMPLE 76

### (3S,4R,5S,6R)-3-(hydroxyamino)-6-(hydroxymethyl)tetrahydro-2H-pyran-2,4,5-triol

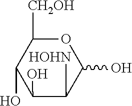

### EXAMPLE 77

### (3R,4R,5S,6R)-3-hydrazinyl-6-(hydroxymethyl)tetrahydro-2H-pyran-2,4,5-triol

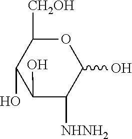

### EXAMPLE 78

### (3S,4R,5S,6R)-3-hydrazinyl-6-(hydroxymethyl)tetrahydro-2H-pyran-2,4,5-triol

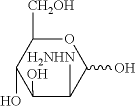

### EXAMPLE 79

### (3R,4S,5S,6R)-6-(hydroxymethyl)-3-mercaptotetrahydro-2H-pyran-2,4,5-triol

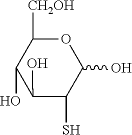

### EXAMPLE 80

### (3S,4S,5S,6R)-6-(hydroxymethyl)-3-mercaptotetrahydro-2H-pyran-2,4,5-triol

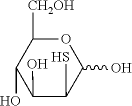

### EXAMPLE 81

### (4R,5S,6R)-3-hydrazono-6-(hydroxymethyl)tetrahydro-2H-pyran-2,4,5-triol

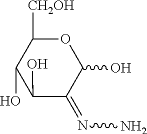

### EXAMPLE 82

### (4R,5S,6R)-2,4,5-trihydroxy-6-(hydroxymethyl)dihydro-2H-pyran-3(4H)-one oxime

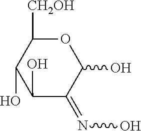

### EXAMPLE 83

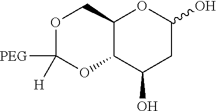

### Anticancer and Antiproliferative Activity

The anticancer and antiproliferative activities of various compounds disclosed herein are disclosed in WO 2009143078 (pages 21-24); WO 2009108926 (paragraphs [0193]-[0197]); WO 2008131024 (paragraphs [0075]-[0088]); and WO 2007101148 (pages 21-37), the disclosures of which are hereby incorporated by reference as if written herein in their entireties.

### Biodistribution and In Vivo Conversion to 2-DG

Analytical methodologies (LC/MS) have been developed that are capable of quantifying concentrations of the compounds of the present invention and/or the liberation of 2-DG in various biomatrices (plasma and various tissues).

CD-1 mice are divided into treatment groups and administered compounds disclosed herein. Individual groups of animals are sacrificed at 0.25, 0.5, 1, 2, 4, and 8 hours following dose administration. From each animal plasma, skin, and other tissues are harvested and analyzed by LC/MS.

### In Vivo Human A375 Melanoma Assay

The compounds disclosed herein can be tested for their ability to decrease tumor size in human A375 melanoma tumors grown in nude mice. For example, see WO 2005058829 (pages 34-35), U.S. Pat. No. 7,745,468 (column 25, line 14 to column 31, line 44), the disclosures of which are hereby incorporated by reference as if written herein in their entireties.

### Human Psoriasis Study

The compounds disclosed herein can be tested for their ability to reduce and/or resolve psoratic lesions in humans. For example, see US 20080167277 (page 3) and WO 2008083389 (page 9), the disclosures of which are hereby incorporated by reference as if written herein in their entireties.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of a dermatological disease comprising the administration, to a patient in need thereof, a therapeutically effective amount of a compound of structural Formula I 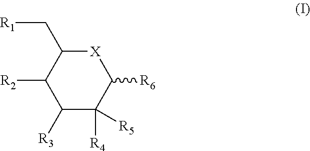
or a salt thereof, wherein:
X is selected from the group consisting of O and S;
R₁, R₂, R₃, and R₆ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, and carbamate, any of which may be optionally substituted;
R₄ and R₅ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, carbamate, alkyl, haloalkyl, perhaloalkyl, —N(R₇)OR₈, —ON(R₉)₂, —N(R₁₀)N(R₁₁)₂, any of which may be optionally substituted, or R₄ and R₅, taken together, are selected from the group consisting of ═N—OR₁₂ and ═N—N(R₁₃)₂; and
R₇, R₈, R₉, R₁₀, R₁₁, R₁₂, and R₁₃ are each independently selected from the group consisting of hydrogen and alkyl, wherein said alkyl may be optionally substituted.

2. The method of claim 1, wherein said dermatological disease is an inflammatory dermatological disease or a proliferative dermatological disease.

3. The method of claim 2, wherein said compound has structural Formula II 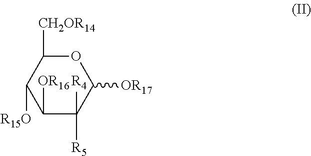
or a salt thereof, wherein:
R₁₄, R₁₅, R₁₆, and R₁₇ are independently selected from the group consisting of hydrogen, COCH₃, COCH₂CH₃, and COCH₂CH₂CH₃; and
R₄ and R₅ are independently selected from the group consisting of hydrogen, Cl, Br, I, ¹⁸F, and ¹⁹F.

4. The method of claim 3, wherein R₄ and R₅ are independently selected from the group consisting of hydrogen, ¹⁸F, and ¹⁹F.

5. The method of claim 4, wherein R₁₄, R₁₅, R₁₆, and R₁₇ are independently selected from the group consisting of hydrogen and COCH₃.

6. The method of claim 2, wherein said compound has structural Formula III or structural Formula IV 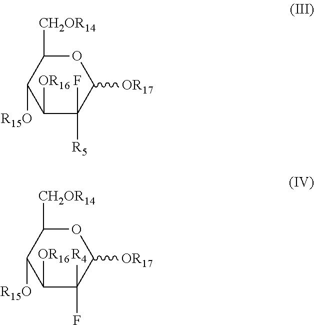
or a salt thereof, wherein:
R₁₄, R₁₅, R₁₆, and R₁₇ are independently selected from the group consisting of hydrogen, COCH₃, COCH₂CH₃, and COCH₂CH₂CH₃;
R₄ and R₅ are independently selected from the group consisting of alkyl, lower alkyl, substituted alkyl, cycloalkyl, hydroxyl, alkoxy, acyl, alkenyl, alkylene, alkylamino, alkylthio, alkylidene, alkynyl, amido, carbamoyl, acylamino, carbamate, O-carbamyl, N-carbamyl, carbonyl, carboxy, carboxylate, ester, ether, halogen, haloalkoxy, haloalkyl, heteroalkyl, hydrazinyl, hydroxyalkyl, isocyanato, isothiocyanato, mercaptyl, nitro, oxy, NH₂, NR₁₈R₁₉, and NHCOR₂₀;
R₁₈ and R₁₉ are selected from the group consisting of hydrogen, alkyl, lower alkyl, substituted alkyl, cycloalkyl, acyl, alkenyl, alkylene, alkylamino, alkylthio, alkylidene, alkynyl, amido, haloalkyl, heteroalkyl, hydrazinyl, and hydroxyalkyl; and
R₂₀ is selected from the group consisting of hydrogen, lower alkyl, substituted lower alkyl, alkenyl, alkylene, alkylamino, alkylthio, alkylidene, alkynyl, amido, carbamoyl, haloalkyl, and heteroalkyl.

7. The method of claim 6, wherein R₁₄, R₁₅, R₁₆, and R₁₇ are hydrogen.

8. The method of claim 2, wherein said compound has structural Formula V 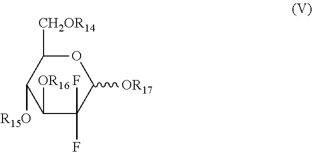
or a salt thereof, wherein:
R₁₄, R₁₅, R₁₆, and R₁₇ are independently selected from the group consisting of hydrogen, COCH₃, COCH₂CH₃, and COCH₂CH₂CH₃.

9. The method of claim 2, wherein said inflammatory dermatological disease or proliferative dermatological disease is selected from the group consisting of psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

10. The method of claim 2, further comprising the administration of another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STATS inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

11. A method of treatment of a dermatological disease comprising the administration, to a patient in need thereof, a therapeutically effective amount of a compound selected from the group consisting of Examples 1 to 83.

12. The method of claim 11, wherein said dermatological disease is an inflammatory dermatological disease or a proliferative dermatological disease.

13. The method of claim 12, wherein said inflammatory dermatological disease or proliferative dermatological disease is selected from the group consisting of psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

14. The method of claim 12, further comprising the administration of another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STATS inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

15. The method of claim 2, wherein said compound has the structural formula:

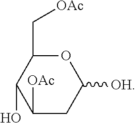

16. The method of claim 15, wherein said inflammatory dermatological disease or proliferative dermatological disease is selected from the group consisting of psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

17. The method of claim 15, further comprising the administration of another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STATS inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

18. A topical pharmaceutical composition for the treatment of a dermatological disease comprising a pharmaceutically acceptable carrier together with a compound of 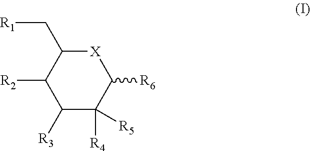
or a salt thereof, wherein:
X is selected from the group consisting of O and S;
R₁, R₂, R₃, and R₆ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, and carbamate, any of which may be optionally substituted;
R₄ and R₅ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, carbamate, alkyl, haloalkyl, perhaloalkyl, —N(R₇)OR₈, —ON(R₉)₂, —N(R₁₀)N(R₁₁)₂, any of which may be optionally substituted, or R₄ and R₅, taken together, are selected from the group consisting of ═N—OR₁₂ and ═N—N(R₁₃)₂; and
R₇, R₈, R₉, R₁₀, R₁₁, R₁₂, and R₁₃ are each independently selected from the group consisting of hydrogen and alkyl, wherein said alkyl may be optionally substituted.

19. The topical pharmaceutical composition of claim 16, wherein said composition is a gel, liniment, lotion, cream, ointment, or paste.

20. The topical pharmaceutical composition of claim 19, wherein said compound has structural Formula II 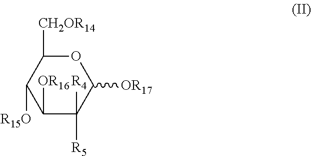
or a salt thereof, wherein:
R₁₄, R₁₅, R₁₆, and R₁₇ are independently selected from the group consisting of hydrogen, COCH₃, COCH₂CH₃, and COCH₂CH₂CH₃; and
R₄ and R₅ are independently selected from the group consisting of hydrogen, Cl, Br, I, ¹⁸F, and ¹⁹F.

21. The topical pharmaceutical composition of claim 20, wherein R₄ and R₅ are independently selected from the group consisting of hydrogen, ¹⁸F, and ¹⁹F.

22. The topical pharmaceutical composition of claim 21, wherein R₁₄, R₁₅, R₁₆, and R₁₇ are independently selected from the group consisting of hydrogen and COCH₃.

23. The topical pharmaceutical composition of claim 19, wherein said compound has structural Formula III or structural Formula IV 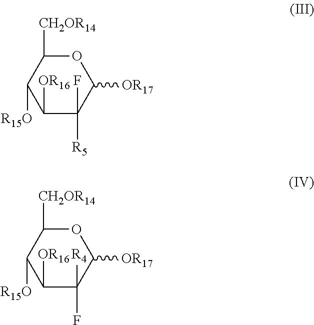
or a salt thereof, wherein:
R₁₄, R₁₅, R₁₆, and R₁₇ are independently selected from the group consisting of hydrogen, COCH₃, COCH₂CH₃, and COCH₂CH₂CH₃;
R₄ and R₅ are independently selected from the group consisting of alkyl, lower alkyl, substituted alkyl, cycloalkyl, hydroxyl, alkoxy, acyl, alkenyl, alkylene, alkylamino, alkylthio, alkylidene, alkynyl, amido, carbamoyl, acylamino, carbamate, O-carbamyl, N-carbamyl, carbonyl, carboxy, carboxylate, ester, ether, halogen, haloalkoxy, haloalkyl, heteroalkyl, hydrazinyl, hydroxyalkyl, isocyanato, isothiocyanato, mercaptyl, nitro, oxy, NH₂, NR₁₈R₁₉, and NHCOR₂₀;
R₁₈ and R₁₉ are selected from the group consisting of hydrogen, alkyl, lower alkyl, substituted alkyl, cycloalkyl, acyl, alkenyl, alkylene, alkylamino, alkylthio, alkylidene, alkynyl, amido, haloalkyl, heteroalkyl, hydrazinyl, and hydroxyalkyl; and
R₂₀ is selected from the group consisting of hydrogen, lower alkyl, substituted lower alkyl, alkenyl, alkylene, alkylamino, alkylthio, alkylidene, alkynyl, amido, carbamoyl, haloalkyl, and heteroalkyl.

24. The topical pharmaceutical composition of claim 23, wherein R₁₄, R₁₅, R₁₆, and R₁₇ are hydrogen.

25. The topical pharmaceutical composition of claim 19, wherein said compound has structural Formula V 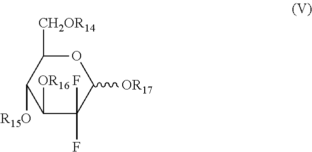
or a salt thereof, wherein:
R₁₄, R₁₅, R₁₆, and R₁₇ are independently selected from the group consisting of hydrogen, COCH₃, COCH₂CH₃, and COCH₂CH₂CH₃.

26. The topical pharmaceutical composition of claim 19, further comprising another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STATS inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

27. A topical pharmaceutical composition for the treatment of a dermatological disease comprising a pharmaceutically acceptable carrier together with a compound selected from the group consisting of Examples 1 to 83.

28. The topical pharmaceutical composition of claim 27, wherein said composition is a gel, liniment, lotion, cream, ointment, or paste.

29. A topical pharmaceutical composition for the treatment of a dermatological disease comprising a pharmaceutically acceptable carrier together with a compound having the structural formula:

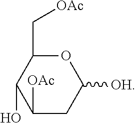

30. The topical pharmaceutical composition of claim 29, wherein said composition is a gel, liniment, lotion, cream, ointment, or paste.

31. The topical pharmaceutical composition of claim 29, further comprising another therapeutic agent selected from the group consisting of cell differentiating agents, anti-proliferative agents, mitochondrial inhibitors, topical steroids, immunosuppressive compounds, JAK2 inhibitors, JAK3 inhibitors, STAT3 inhibitors, STATS inhibitors, HIP-1a inhibitors, parathyroid hormone-related protein (PTHrP) agonists, cell adhesion blockers, non-steroidal anti-inflammatory agents, antibacterial agents, alkylating agents, anti-metabolite agents, mitotic inhibitors, tyrosine kinase inhibitors, topoisomerase inhibitors, cancer immunotherapy monoclonal antibodies, anti-tumor antibiotic agents, anti-cancer agents, autophagy-inducing agents, anti-psoriasis drugs, and D-mannose.

32. A compound for use in the manufacture of a medicament for the prevention or treatment of a dermatological disease, having structural Formula I 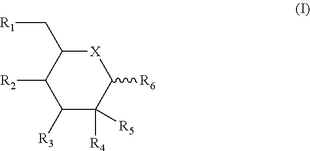
or a salt thereof, wherein:
X is selected from the group consisting of O and S;
R₁, R₂, R₃, and R₆ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, and carbamate, any of which may be optionally substituted;
R₄ and R₅ are independently selected from the group consisting of hydrogen, hydroxyl, thiol, halogen, alkoxy, haloalkoxy, per haloalkoxy, alkoxyalkyloxy, —OC(O)alkyl, OCO₂alkyl, alkylthio, amino, alkylamino, N-sulfonamido, N-amido, carbamate, alkyl, haloalkyl, perhaloalkyl, —N(R₇)OR₈, —ON(R₉)₂, —N(R₁₀)N(R₁₁)₂, any of which may be optionally substituted, or R₄ and R₅, taken together, are selected from the group consisting of ═N—OR₁₂ and ═N—N(R₁₃)₂; and
R₇, R₈, R₉, R₁₀, R₁₁, R₁₂, and R₁₃ are each independently selected from the group consisting of hydrogen and alkyl, wherein said alkyl may be optionally substituted.

33. The compound of claim 32, wherein said dermatological disease is an inflammatory dermatological disease or a proliferative dermatological disease.

34. The compound of claim 33, wherein said compound has structural Formula II 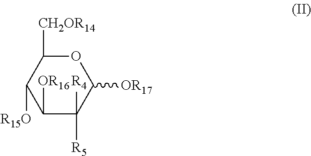
or a salt thereof, wherein:
R₁₄, R₁₅, R₁₆, and R₁₇ are independently selected from the group consisting of hydrogen, COCH₃, COCH₂CH₃, and COCH₂CH₂CH₃; and
R₄ and R₅ are independently selected from the group consisting of hydrogen, Cl, Br, I, ¹⁸F, and ¹⁹F.

35. The compound of claim 34, wherein R₄ and R₅ are independently selected from the group consisting of hydrogen, ¹⁸F, and ¹⁹F.

36. The compound of claim 35, wherein R₁₄, R₁₅, R₁₆, and R₁₇ are independently selected from the group consisting of hydrogen and COCH₃.

37. The compound of claim 33, wherein said compound has structural Formula III or structural Formula IV 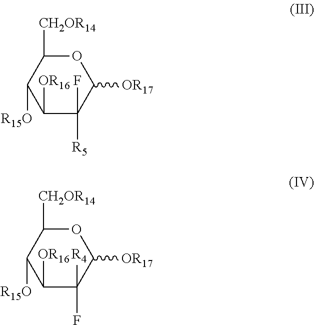
or a salt thereof, wherein:
R₁₄, R₁₅, R₁₆, and R₁₇ are independently selected from the group consisting of hydrogen, COCH₃, COCH₂CH₃, and COCH₂CH₂CH₃;
R₄ and R₅ are independently selected from the group consisting of alkyl, lower alkyl, substituted alkyl, cycloalkyl, hydroxyl, alkoxy, acyl, alkenyl, alkylene, alkylamino, alkylthio, alkylidene, alkynyl, amido, carbamoyl, acylamino, carbamate, O-carbamyl, N-carbamyl, carbonyl, carboxy, carboxylate, ester, ether, halogen, haloalkoxy, haloalkyl, heteroalkyl, hydrazinyl, hydroxyalkyl, isocyanato, isothiocyanato, mercaptyl, nitro, oxy, NH₂, NR₁₈R₁₉, and NHCOR₂₀;
R₁₈ and R₁₉ are selected from the group consisting of hydrogen, alkyl, lower alkyl, substituted alkyl, cycloalkyl, acyl, alkenyl, alkylene, alkylamino, alkylthio, alkylidene, alkynyl, amido, haloalkyl, heteroalkyl, hydrazinyl, and hydroxyalkyl; and
R₂₀ is selected from the group consisting of hydrogen, lower alkyl, substituted lower alkyl, alkenyl, alkylene, alkylamino, alkylthio, alkylidene, alkynyl, amido, carbamoyl, haloalkyl, and heteroalkyl.

38. The compound of claim 37, wherein R₁₄, R₁₅, R₁₆, and R₁₇ are hydrogen.

39. The compound of claim 33, wherein said compound has structural Formula V 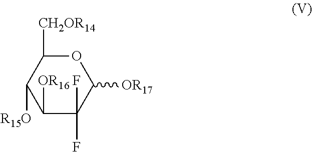
or a salt thereof, wherein:
R₁₄, R₁₅, R₁₆, and R₁₇ are independently selected from the group consisting of hydrogen, COCH₃, COCH₂CH₃, and COCH₂CH₂CH₃.

40. The compound of claim 33, wherein said inflammatory dermatological disease or proliferative dermatological disease is selected from the group consisting of psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.

41. A compound for use in the manufacture of a medicament for the prevention or treatment of a dermatological disease, selected from the group consisting of Examples 1 to 83.

42. The compound of claim 41, wherein said dermatological disease is an inflammatory dermatological disease or a proliferative dermatological disease.

43. A compound for use in the manufacture of a medicament for the prevention or treatment of a dermatological disease, wherein said compound has the structural formula:

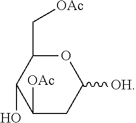

44. The compound of claim 43, wherein said dermatological disease is an inflammatory dermatological disease or a proliferative dermatological disease.

45. The compound of claim 44, wherein said inflammatory dermatological disease or proliferative dermatological disease is selected from the group consisting of psoriasis, plaque psoriasis, psoriasis vulgaris, localized pustular psoriasis, pustule psoriasis, Hallopeau localized continuous achrodermatitis, pustular palm psoriasis, pustular sole psoriasis, generalized pustular psoriasis, von Zumbuch generalized pustular psoriasis, milia psoriasis, Hallopeau generalized continuous dermatitis, herpetiform impetigodermatitis, atopic dermatitis, seborrheic dermatitis, contact dermatitis, numular dermatitis, generalized exfoliative dermatitis, statis dermatitis, perioral dermatitis, acne, rosacea, boils, carbuncles, pemphigus, cellulitis, Grover's disease, hidradenitis suppurativa, lichen planus, chronic lichen simplex, rhinophyma, pseudofolliculitis barbae, inflammatory reactions, drug eruptions, erythema, erythema multiforme, erythema nodosum, granuloma annulare, eczema, xerosis, terosis, ichthyosis, epidermolytic hyperkeratosis, keratoses, pruritis, cradle cap, scales, fresh stretch marks, dermatoses, burns, skin hypersensitivity reactions (including poison ivy and poison oak), decubitus ulcers, pressure ulcers, diabetic ulcers, epidermolysis bullosa, eczematoid dermatitis, pemphigus, bullous pemphigoid, epidermolysis bullosa, urticaria, angioedema, vasculitides, dermal eosinophilia, vitiligo, alopecia greata, skin cancers, cutaneous T cell lymphoma, basal cell carcinoma, nodular basal cell carcinoma, cystic basal cell carcinoma, cicatricial basal cell carcinoma, infiltrative basal cell carcinoma, Micronodular basal cell carcinoma, superficial basal cell carcinoma, pigmented basal cell carcinoma, Jacobi ulcer, fibroepithelioma of Pinkus, polypoid basal cell carcinoma, pore-like basal cell carcinoma, aberrant basal cell carcinoma, squamous cell carcinoma, adenoid squamous cell carcinoma, clear cell squamous cell carcinoma, spindle cell squamous cell carcinoma, signet-ring cell squamous cell carcinoma, basaloid squamous cell carcinoma, verrucous carcinoma, keratoacanthoma, Bowen's disease, Marjolin's ulcer, melanoma, lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, soft-tissue melanoma, dermatofibrosarcoma protuberans, Merkel cell carcinoma, Kaposi's sarcoma, spindle cell tumors, sebaceous carcinomas, microcystic adnexal carcinoma, Pagets's disease, atypical fibroxanthoma, leimyosarcoma, angiosarcoma, and Merkel cell carcinoma.
